(19) United States
(12) Patent Application Publication
(10) Pub. No.: US 2015/0065777 A1
(43) Pub. Date: Mar. 5, 2015

(54) RADIATION THERAPY MACHINE WITH REAL-TIME EBT IMAGING

(71) Applicant: Naser Darwish, Chicago, IL (US)

(72) Inventor: Naser Darwish, Chicago, IL (US)

(21) Appl. No.: 14/013,720

(22) Filed: Aug. 29, 2013

(51) Int. Cl. A61N 5/10

(52) U.S. Cl. CPC A61N 5/1081
USPC 600/1; 378/65

(57) ABSTRACT

The present invention provides a radiation therapy machine using electron beam tomography to provide rapid real-time measurement of radiation therapy target movement. The electron beam therapy may be mounted on a gantry to move with the radiation therapy head or both the megavoltage and kilovoltage radiation needed for radiation therapy and tomography may be implemented by a single combined electron beam tube.

### STATEMENT REGARDING FEDERALLY SPONSORED RESEARCH OR DEVELOPMENT

——

### CROSS REFERENCE TO RELATED APPLICATION

——

### BACKGROUND OF THE INVENTION

The present invention relates to radiotherapy equipment and in particular to a radiotherapy machine providing concurrent real-time imaging.

Medical equipment for radiation therapy treats tumorous tissue with high-energy radiation. The amount of radiation and its placement must be accurately controlled to ensure both that the tumor receives sufficient radiation to be destroyed, and that the damage to the surrounding and adjacent non-tumorous tissue is minimized.

In external source radiation therapy, a radiation source external to the patient treats internal tumors. The external source is normally collimated to direct a beam only to the tumorous site. The source of high-energy radiation may be x-rays, or electrons from linear accelerators in the range of 2-25 MeV, or gamma rays from highly focused radioisotopes such as a Co⁶⁰ source having an energy of 1.25 MeV.

Typically, the tumor will be treated from several different angles with the intensity and shape of the beam adjusted appropriately. The purpose of using multiple beams which converge on the site of the tumor is to reduce the dose to areas of surrounding non-tumorous tissue. The angles at which the tumor is irradiated are selected to avoid angles which would result in irradiation of particularly sensitive structures near the tumor site. The angles and intensities of the beams for a particular tumor form a treatment plan for that tumor.

One highly accurate method of controlling the dose to a patient termed intensity modulated radiation therapy (IMRT) employs a radiation source that produces a fan beam composed of many individual rays whose intensity may be independently controlled. The fan beam orbits the patient within a plane illuminating a slice of the patient, while the intensity of each ray of the fan beam is modulated as a function of that angle. By properly selecting the beam intensities at different angles, complex regions within the slice may be accurately irradiated. U.S. Pat. No. 5,317,616, describes the construction of one such machine and one method of calculating the necessary beam intensities as a function of angle.

In order to take advantage of the improved accuracy in dose placement offered by such radiation therapy systems, the radiation treatment plan may be based on a computed tomography (“CT”) image of the patient. The CT image is obtained on a dedicated machine providing a gantry with an x-ray tube that may rotate about the patient collecting fan beam projections at multiple angles. Using the CT image, the radiologist and/or the radiation oncologist views the tumorous area and determines the beam angles and intensities (identified with respect to the tumor image) which will be used to treat the tumor. In an automated system, a computer program selects the beam angles and intensities after the physician identifies the tumorous region and upper and lower dose limits for the treatment.

Normally, the CT image of the patient is acquired substantially before the radiation treatment occurs to allow time for the treatment plan to be prepared. As a result, the patient may have moved in position in between the tune of the CT image acquisition and the radiation treatment. This will also be true in cases where the treatment occurs during a number of different treatment sessions over time. In addition, normal physiological processes may cause movement of the tumor or other tissue during a radiation treatment or between treatment sessions.

Uncertainty in the positioning of the patient with respect to the original CT image can defeat much of the accuracy gains expected from the use of a CT image for treatment planning.

Combining a radiation therapy with the CT machine has been proposed with respect to reducing registration problems occurring between obtaining the CT planning images and performing the radiation therapy. Such a CT system would provide little utility with respect to correcting for patient movement during radiation therapy. Further, normally the CT machine is mounted to project x-rays perpendicularly to the megavoltage radiation to eliminate interference. For this reason, information about patient movement in the treatment plane of the megavoltage radiation source may be delayed until after the megavoltage radiation source and CT radiation source have moved at least 90° eliminating the timeliness and reducing accuracy of the measurement. In addition, collection of the CT image normally requires approximately 180° of CT imaging requiring movement of the radiation therapy source among a range of positions before accurate position data may be obtained.

### SUMMARY OF THE INVENTION

The present invention combines a megavoltage radiation source with an electron beam CT to rapidly obtain in-plane and cross-plane kilovoltage x-ray information at each orientation of the megavoltage radiation source. To the extent that the CT images are used for detecting movement of the patient for radiation therapy, extremely high-speed, lower resolution CT from fast electron beam tomography (EBT) imaging may be used. Cone beam imaging may be implemented to obtain volumetric data at extremely high data rates.

Specifically, the invention provides a radiotherapy machine having a first arcuate target extending at least substantially 180° about an axis along which a patient table maybe received. An electron accelerator assembly is displaced along the axis away from the arcuate target and steerable to direct a first beam of electrons at different portions of the arcuate target to direct x-rays substantially along the plane perpendicular to the axis. An evacuated chamber holds the arcuate or planar target and electron accelerator assembly. An arcuate or planar detector system is positioned in opposition with the arcuate target about the axis to receive x-rays from the arcuate target and a therapeutic radiation source directs high-energy therapeutic radiation along the plane.

It is thus a feature of at least one embodiment of the invention to provide a high-speed tomographic system permitting rapid response to tumor movement or change during radiation therapy. The EBT system allows a flexible trade-off between image resolution and acquisition speed allowing dual use for high-resolution image and real-time motion tracking correction.

The radiotherapy machine may further include a gantry for movement of the arcuate or planar target and detector rotationally about the axis.

It is thus a feature of at least one embodiment of the invention to reduce the size of the EBT system by allowing it to be moved about the patient.

The radiotherapy machine may further include a modulator for providing a set of beam portions having independently controllable strength.

It is thus a feature of at least one embodiment of the invention to provide a position tracking system better matched to the high-resolution radiation therapy obtained with multi-leaf collimator-type systems.

The therapeutic radiation source may be on a gantry arm displaced from the first arcuate or planar target and movable with the gantry.

It is thus a feature of at least one embodiment of the invention to allow the EBT system to track the therapeutic radiation source with gantry movement to simplify position tracking by using a reference frame consistent with the therapeutic radiation source.

The therapeutic radiation source may direct the high-energy therapeutic radiation along an axis substantially parallel to a segment within the plane between the ends of the arcuate or planar target.

It is thus a feature of at least one embodiment of the invention to provide an EBT system that obtains in-plane and cross-plane motion data for all orientations of the therapeutic radiation source.

In an alternative embodiment, the therapeutic radiation source may be formed of a second beam of electrons of higher energy than the first beams of electrons directed at the arcuate or planar target to produce high-energy radiation.

It is thus a feature of at least one embodiment of the invention to provide an integrated electron beam therapy and radiation beam system providing extremely versatile and accurate radiation therapy.

The arcuate target may provide a high- and low-energy portion wherein the first beam of electrons may be directed toward the low-energy portion and the second beam of electrons district to the high-energy portion.

It is thus a feature of at least one embodiment of the invention to optimize the targets for the production of high- and low-energy radiation.

These particular objects and advantages may apply to only some embodiments falling within the claims and thus do not define the scope of the invention.

### BRIEF DESCRIPTION OF THE DRAWINGS

FIG. 1 is a simplified perspective view of a radiation therapy machine of the present invention showing a gantry-mounted high-energy radiation source with a multi-leaf collimator positioned adjacent to an electron beam tube and detector for electron beam tomography;

FIG. 2 is a block diagram of the elements of FIG. 1 showing control of these elements with a central controller;

FIG. 3 is a timing diagram showing energizing of the high-energy therapeutic radiation and lower-energy imaging radiation in between measurement periods;

FIG. 4 is a figure similar to FIG. 1 showing integration of the high-energy radiation source with the electron beam tube;

FIG. 5 is a schematic representation of a twin target system for producing high-energy and lower-energy radiation in a single electron beam tube; and

FIG. 6 is a front elevational view of a radiation therapy machine incorporating PET detectors.

### DETAILED DESCRIPTION OF THE PREFERRED EMBODIMENT

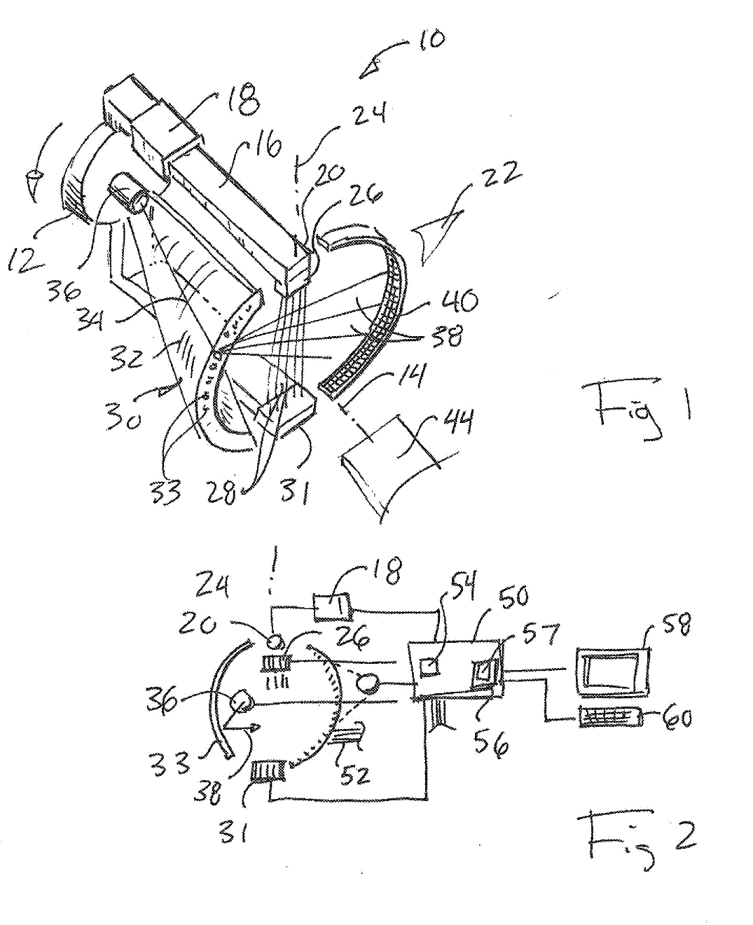

Referring now to FIG. 1, a radiation therapy machine 10 according to one embodiment of the present invention may provide for a rotatable gantry 12 rotating about an axis 14. The gantry 12 may support an axially extending gantry arm 16 supporting a megavoltage accelerator 18, for example a linear accelerator (LINAC), directing high-energy electrons to a radiation head 20 at an imaging/treatment plane 22.

The radiation head 20 may provide a target (not shown) directing high-energy megavoltage radiation along the plane 22 perpendicular to the axis 14 along the treatment axis 24. The high-energy megavoltage radiation is received by a multi-leaf collimator 26 to produce a variety of independently modulated megavoltage beams 28 of a type known in the art. The modulated megavoltage beams 28 then cross the axis 14 to be received by portal imaging device 31 also mounted for movement on the gantry.

To the side of the radiation head 20, an arcuate electron beam tube 30 is positioned providing an evacuated housing 32 moving with the gantry 12. Supported within the evacuated housing 32 at the imaging/treatment plane 22 and lying within the imaging/treatment plane 22 is arcuate target anode 33 ideally extending a constant radius about axis 14. Ends of the arcuate target anode 33 define a segment generally parallel to treatment axis 24.

The target anode 33 may receive a kilovoltage electron beam 34 from an electron accelerator assembly 36. The electron accelerator assembly 36 includes an electron source and focusing and scanning magnets as is understood in the art so that the electron beam 34 may be scanned in an arc at high speed across the target anode 33 to produce cone beam kilovoltage beams 38 to cross treatment axis 24 from a range of angles of approximately 180 degrees about axis 14.

The kilovoltage beams 38 are received by a detector array 40 generally being an opposed arcuate array mirroring the target anode 33. Ideally the detector array 40 will provide two dimensions of cone beam radiation sensing. A general construction of the arcuate electron beam tube 30 and detector array 40 is provided in U.S. Pat. Nos. 5.633,906, and 7,688,937 hereby incorporated by reference.

A patient table 44 may support a patient (not shown) to be received along the axis 14 into the imaging/treatment plane 22 between the target anode 33 and detector array 40 and between the radiation head 20 and portal-imaging device 31. In use, the radiation head 20 may be rotated about the patient on the patient table 44 to provide high-resolution IMRT radiation therapy. At each such position, the electron beam 34 may be scanned through approximately 180° of the target anode 33 to obtain a CT reconstruction allowing accurate determination of the position of a tumor or the like being treated by the megavoltage beams 28.

Referring now to FIG. 2, this operation will generally be under the control of the controller computer 50 connecting to the megavoltage accelerator 18, the portal imaging device 31 and the multi-leaf collimator 26 to implement a radiation therapy plan, for example, defining positions of the multi-leaf collimator leaves at each angle of the radiation treatment axis 24. The computer 50 may also control movement of the gantry 12 by means of a motor and encoder assembly.

Computer 50 will also provide signals to the electron accelerator assembly 36 to control the scanning and acquisition of CT data received over data lines 52 by the computer 50 for CT reconstruction. As is generally understood in the art, the computer 50 may include a processor 54 and a memory 56, the latter holding a program 57 for implementing the radiation therapy plan and reconstructing the CT image and providing adjustments in the radiation therapy plan in real-time according to movement or shape-changes of the tissue being treated as detected in the CT images. Alternatively or in addition, the movement of the patient table 44 may be adjusted to realign the tumor with the radiation treatment plan according to the CT images. Techniques for adjusting a radiation treatment plan based on patient movement is described, for example, in U.S. Pat. No. 5,673,300 hereby incorporated by reference.

Output of the radiation therapy program in the form of cumulative dose derived from the portal imaging device 31 and the current CT images may be displayed on a computer monitor 58 and commands from the user with respect to controlling the process may be received by various user interface devices 60 such as a keyboard and/or mouse.

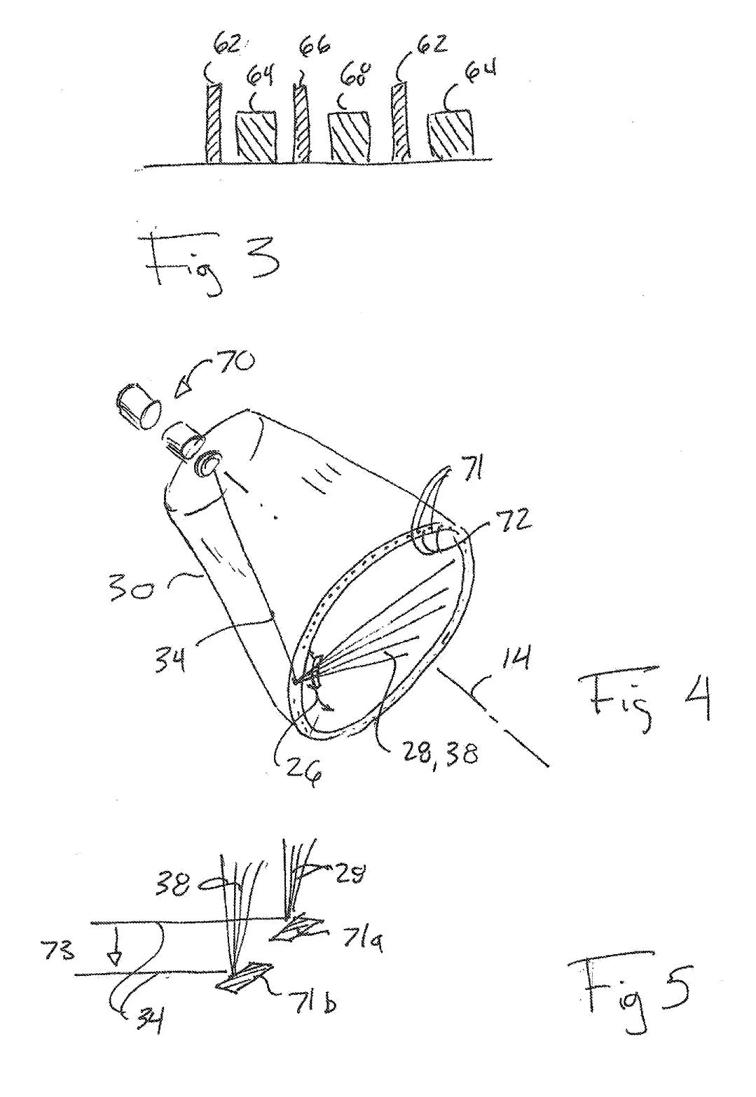

Referring now to FIG. 3, during a scanning process, kilovoltage beams 38 may be produced at a first interval 62 followed by a data acquisition and processing time 64 followed by megavoltage beam 28 as indicated by interval 66 followed by data reconstruction 68 using portal imaging device 31. The interleaving of kilovoltage beams 38 and megavoltage beams 28 in time substantially reduces interference between the two beams with respect to receipt of scattered megavoltage radiation by detector array 40 and scattered kilovoltage radiation by portal imaging device 31.

Referring now to FIG. 4, in an alternative embodiment, a single electron accelerating source 70 may be used serving to produce the megavoltage electrons provided by megavoltage accelerator 18 and the kilovoltage electrons provided by the electron accelerator assembly 36 described above. In this case, the accelerating source 70 may be a LINAC, which allows the output energy to be rapidly switched from high energy to low energy. This switching may control the phase of the radio frequency (RF) power used by the LINAC to switch it by 180 degrees after initial acceleration of the electron beam to decelerate the beam, rather than accelerating it, converting megavoltage electrons into kilovoltage electrons. The LINAC cavity can be detuned to variably change the phase of the radio frequency energy reflected from the output coupling cavity so that regions of the accelerator can be selectively turned off when one of the intermediate tunable coupling cavities is detuned. Energy output of the electron downstream of the accelerating structure can be adjusted for the level of microwave power by varying design parameters of the main bundler cavities or side cavities. Cavities can be detuned to variably change the phase of the radio frequency energy reflected from the output coupling cavity so that regions of the accelerator can be selectively turned off when one of the intermediate tunable coupling cavities is detuned.

To go from higher to lower energy, the standing wave at a downstream portion of the standing wave LINAC can be disrupted so that less acceleration acts on the electron beam. An energy switch positioned in a side cavity can be a mechanical switch according to U.S. Pat. No. 4,629,938 or an electronic switch according to U.S. Pat. No. 7,112,924 both hereby incorporated by reference. The switch can be used to disrupt the resonant coupling between two neighboring main cavities. The buncher cavities function to accelerate the bunch of electrons appropriately to ride at or near the crest of the electromagnetic wave in the accelerating main cavities of the LINAC. If the power of the electromagnetic field is modified, the bunch does not ride at or near the crest of the electromagnetic wave and the electrons would be decelerated based on the specified phase shift.

In an alternative embodiment, the second electron accelerator assembly (not shown) may be fed into a distinct accelerating structure or voltage difference sufficient for generation of kilovoltage x-rays but synchronized to generate kilovoltage x-rays during the pulse off-period of the radiotherapy MV beam. An introduction of a second source of electrons may shield the main electron accelerator assembly or a solid-state electron emitter may include carbon nanotubes that may control the output of the of the electron beam to synchronize with the radiotherapy MV beam. The second electron accelerator assembly may modulate the intensity of electrons emitted by the first electron accelerator assembly to decrease the output of the electrons. A portion of the electron accelerator assembly output of the LINAC can also be used to be accelerated in another set of accelerating structures applicable for the lower energy range such as kilovoltage range applicable for computed tomographic imaging.

In the embodiment of FIG. 4, the target anode 33 maybe arcuate as shown in FIG. 1 or may encompass an entire circle to provide a circular anode array 71. The elements of the circular anode array 71 maybe interleaved with detector elements 72. At each position of the electron beam 34 around the circular anode array 71, as may be scanned in an entire circle around axis 14, the accelerating source 70 may switch between megavoltage and kilovoltage control. A mechanically mounted multi-leaf collimator 26 may be moved in synchrony with the electron beam 34 to provide for collimation during the activation of the megavoltage radiation per the schedule shown in FIG. 3.

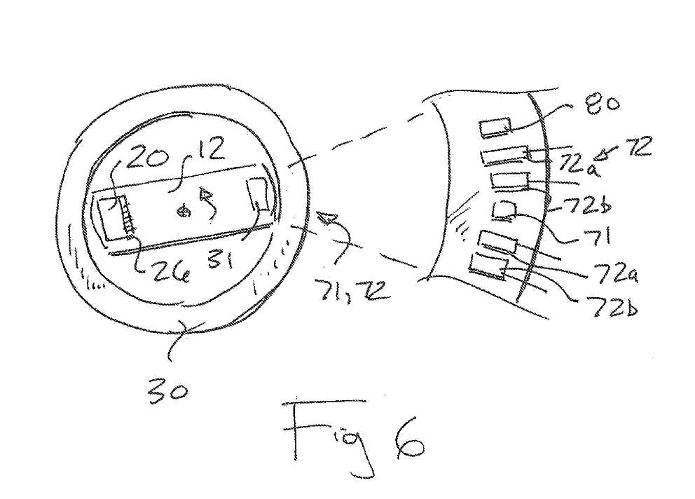

Referring now to FIG. 5, in one embodiment a separate set of coaxial circular anode arrays 71a and 71b may be used to produce the megavoltage beams 26 and kilovoltage beams 38 to optimize the anodes for this different purpose by simple radial adjustment indicated by arrow 73 of the electron beam 34 to select among these different targets,

Generally the electron beam may be controlled according to profile, position, and orientation of the electron beam spot to conform to the desired profile, position, and orientation of the beam spot at various positions along the X-ray-producing target, which can be positioned in a ring, dual-ring or open-ring geometry, arcs in pairs or triplets, or dual-head geometry. A main line of electron beams can be split for each ring in the pair, each are in the pair, or flat head in the pair. A distinct set of electron beams can also generate x-rays for each component of the pair or triplets. Either part of the pair can act as a radiation detection side. Furthermore, the detectors can be interleaved on the same side of the radiation source or on both sides if the radiation source is originating from both sides.

Referring now to FIG. 6, the embodiment of FIG. 4 may be augmented by the use of PET detectors 72a interleaved with x-ray detectors 72b within the electron beam tube 30 or positioned closely concentric to the anodes 71, where the PET detectors 71a are capable of also detecting positron emission so that positron emission tomography (PET) is integrated with any form of real time onboard CT. In this case conventional anodes 71 maybe employed in the electron beam tube 30 or cold cathode x-ray generators 80 arrayed in an arc. In either case, x-rays sources and KV and PET detectors are interleaved in detector pairs or triplets, single or multiple detector arcs, or single or multiple detector rings. The combination of physiological based imaging using PET with anatomical and soft tissue imaging using KV CT with either electron beam CT or cold cathode CT brings a unique marriage to linear accelerators. in such fashion, a tumor response to radiation is detected with PET and tumor treatment is improved due to precise and accurate positioning of the tumor in real time during the radiation treatment session. In this regard, program 57 may further reconstruct PET images using this data and the separate radiation head 20 and portal monitor 31 may be mounted on the gantry 12 for rotation independent of the electron beam tube 30. Generally, it will be appreciated that the electron beam tube 30 may be stationary or may be rotated with the gantry 12 as has been described.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a microprocessor” and “a processor” or “the microprocessor” and “the processor,” can be understood to include one or more microprocessors that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

I claim:

1. A radiotherapy machine comprising:
a first arcuate target extending at least substantially 180° within a plane perpendicular o an axis along which a patient table maybe received;
an electron accelerator assembly displaced along the axis away from the arcuate target and steerable to direct a first beam of electrons at different portions of the arcuate target to direct x-rays substantially along the plane perpendicular to the axis;
an evacuated chamber holding the arcuate target and electron accelerator assembly;
an arcuate detector system in opposition with the arcuate target about the axis to receive x-rays from the arcuate target; and
a therapeutic radiation source directing high-energy therapeutic radiation along the plane.

2. The radiotherapy machine of claim 1 further including a gantry for movement of the arcuate target and detector rotationally about the axis.

3. The radiotherapy machine of claim 1 wherein the therapeutic radiation source further includes a modulator for providing a set of beam portions having independently controllable strength.

4. The radiotherapy machine of claim 2 further including the therapeutic radiation source on a gantry arm displaced from the first arcuate target and movable with the gantry.

5. The radiotherapy machine of claim 1 wherein the therapeutic radiation source directs the high-energy therapeutic radiation along an axis substantially parallel to a segment within the plane between ends of the arcuate target.

6. The radiotherapy machine of claim 1 wherein the x-rays are kilovoltage x-rays.

7. The radiotherapy machine of claim 1 wherein the high-energy radiation is megavoltage x-rays.

8. The radiotherapy machine of claim 1 wherein the megavoltage radiation source comprises a second beam of electrons of higher energy than the first beams of electrons directed at the arcuate target to produce high-energy radiation.

9. The radiotherapy machine of claim 8 wherein the arcuate target provides a high- and low-energy portion and wherein the first beam of electrons is directed toward the low-energy portion and the second beam of electrons is directed to the high-energy portion.

10. The radiotherapy machine of claim 8 wherein the x-rays are kilovoltage x-rays.

11. The radiotherapy machine of claim 8 wherein the high-energy radiation is megavoltage x-rays.

12. The radiotherapy machine of claim 1 wherein the arcuate detector system in opposition with the arcuate target further includes PET detectors interlaced with x-ray detectors.
